SEQUENCE LISTING (30 sequences)

SEQ ID NO: 1 - DNA, 67 bp, Artificial sequence
  Synthetic construct
cgcgtccccggacttatcaaactggtttttcaagagaaaaccagtttgataagtcctttttggaaat

SEQ ID NO: 2 - DNA, 65 bp, Artificial sequence
  Synthetic construct
cgatttccaaaaacctgaatagtttgaccaaaagagaactttttggtcaaactattcaggcccct

SEQ ID NO: 3 - DNA, 21 bp, Artificial sequence
  Synthetic construct
ggacuuaucaaacugguuutt

SEQ ID NO: 4 - DNA, 21 bp, Artificial sequence
  Synthetic construct
aaaccaguuugauaagucctt

SEQ ID NO: 5 - DNA, 27 bp, Artificial sequence
  Synthetic construct
gaattcgtcgacgaggttagctctttc

SEQ ID NO: 6 - DNA, 25 bp, Artificial sequence
  Synthetic construct
gaattctctagagccaccgtccagg

SEQ ID NO: 7 - DNA, 37 bp, Artificial sequence
  Synthetic construct
gctagcatgccgcctaaggacgacaagaagaagaagg

SEQ ID NO: 8 - DNA, 48 bp, Artificial sequence
  Synthetic construct
ttgtctttcttggccgactttccagcgtccttcttcttcttgtcgtcc

SEQ ID NO: 9 - DNA, 47 bp, Artificial sequence
  Synthetic construct
gaaagtcggccaagaaagacaaagacccagtgaacaaatccgggggc

SEQ ID NO: 10 - DNA, 47 bp, Artificial sequence
  Synthetic construct
gcctttggaccacttcttctttttggccttgcccccggatttgttca

SEQ ID NO: 11 - DNA, 51 bp, Artificial sequence
  Synthetic construct
aaaagaagaagtggtccaaaggcaaagttcgggacaagctcaataacttag

SEQ ID NO: 12 - DNA, 53 bp, Artificial sequence
  Synthetic construct
ttatcataggtagctttgtcaaacaagactaagttattgagcttgtcccgaac

SEQ ID NO: 13 - DNA, 53 bp, Artificial sequence
  Synthetic construct
tcttgtttgacaaagctacctatgataaactctgtaaggaagttcccaactat

SEQ ID NO: 14 - DNA, 50 bp, Artificial sequence
  Synthetic construct
gagaccacagctggggttataagtttatagttgggaacttccttacagag

SEQ ID NO: 15 - DNA, 47 bp, Artificial sequence
  Synthetic construct
tataaccccagctgtggtctctgagagactgaagattcgaggctccc

SEQ ID NO: 16 - DNA, 45 bp, Artificial sequence
  Synthetic construct
gagctcctgaagggctgccctggccagggagcctcgaatcttcag

SEQ ID NO: 17 - DNA, 44 bp, Artificial sequence
  Synthetic construct
cagcccttcaggagctccttagtaaaggcctgattaagctcgtg

SEQ ID NO: 18 - DNA, 48 bp, Artificial sequence
  Synthetic construct
ggtgtaaattacttgagctctgtgctttgacacgagcttaatcaggcc

SEQ ID NO: 19 - DNA, 49 bp, Artificial sequence
  Synthetic construct
cacagagctcaagtaatttacaccagaaataccaagggtggagatgctc

SEQ ID NO: 20 - DNA, 48 bp, Artificial sequence
  Synthetic construct
ggatcctcatgcatcttcaccagcagctggagcatctccacccttggt

SEQ ID NO: 21 - DNA, 17 bp, Artificial sequence
  Synthetic construct
gctagcatgccgcctaa

SEQ ID NO: 22 - DNA, 18 bp, Artificial sequence
  Synthetic construct
atgccgcctaaggacgac

SEQ ID NO: 23 - DNA, 18 bp, Artificial sequence
  Synthetic construct
gcactcttccagccttcc

SEQ ID NO: 24 - DNA, 66 bp, Artificial sequence
  Synthetic construct
cgcgtccccggacttatcaaactggtttttcaagagaaaaccagtttgataagtcctttttggaaa

SEQ ID NO: 25 - DNA, 19 bp, Artificial sequence
  Synthetic construct
ccatggtacccggttcctc

SEQ ID NO: 26 - DNA, 26 bp, Artificial sequence
  Synthetic construct
ccatggggcagtgacccggaaggcag

SEQ ID NO: 27 - DNA, 21 bp, Artificial sequence
  Synthetic construct
ggatcctcatgcatcttcacc

SEQ ID NO: 28 - DNA, 18 bp, Artificial sequence
  Synthetic construct
tcatgcatcttcaccagc

SEQ ID NO: 29 - DNA, 19 bp, Artificial sequence
  Synthetic construct
gcgctcaggagggagcaat

SEQ ID NO: 30 - DNA, 65 bp, Artificial sequence; identical to SEQ ID NO: 2
  Synthetic construct

(19) United States
(12) Patent Application Publication
(10) Pub. No.: US 2015/0105433 A1
(43) Pub. Date: Apr. 16, 2015

(54) TREATING VIRAL INFECTIONS HAVING VIRAL RNAs TRANSLATED BY A NON-IRES MEDIATED MECHANISM

(71) Applicant: THE UAB RESEARCH FOUNDATION, Birmingham, AL (US)

(72) Inventor: Sunnie R. Thompson, Birmingham, AL (US)

(21) Appl. No.: 14/397,396

(22) PCT Filed: Apr. 25, 2013
(86) PCT No.: PCT/US13/38181

§ 371 Date: Oct. 27, 2014

Related U.S. Application Data
(60) Provisional application No. 61/639,179, filed on Apr. 27, 2012.

(51) Int. Cl. C07D 277/46; C07C 235/56; C12Q 1/68; C07C 219/28; C07D 231/12; C07D 213/74; C07D 213/52

(52) U.S. Cl. CPC C07D 277/46; C07D 213/74; C07C 235/56; C07D 213/52; C07C 219/28; C07D 231/12; C12Q 1/6876; C12Q 2600/158
USPC 514/352; 514/622; 514/371; 514/357; 514/654; 514/406; 435/375; 435/29; 435/7.1; 435/7.92; 506/7; 506/9; 435/6.11; 435/6.12; 435/455

(57) ABSTRACT

Provided herein are methods for preventing or treating a viral infection in a subject, wherein the viral infection is mediated by a virus comprising one or more viral RNA molecules translated by a ribosomal shunting mechanism or a non-IRES mediated mechanism. The methods comprise administering to a subject an agent that reduces ribosomal protein (Rps25) expression or function. Also provided are methods of inhibiting or promoting ribosomal shunting-mediated translation or non-IRES mediated translation. Also provided are methods of screening for an agent that inhibits or promotes ribosomal shunting-mediated translation or non-IRES mediated translation.

This application claims the benefit of U.S. Provisional Application No. 61/639,179, filed Apr. 27, 2012, which is hereby incorporated herein in its entirety.

### STATEMENT REGARDING FEDERALLY FUNDED RESEARCH

This invention was made with government funding under Grant Nos. RO1GM084547 and RO1GM084547-01A1S1 from the National Institutes of Health. The government has certain rights in this invention.

### BACKGROUND

The vast majority of messenger RNAs (mRNAs) are translated using a cap-dependent mechanism of translation. However, 5-10% of messages initiate translation using a cap-independent mechanism that is not as well defined. Certain cellular and viral mRNAs are capable of initiating translation using a ribosomal shunting mechanism or other non-IRES mediated mechanisms.

### SUMMARY

Provided herein are compounds and methods for use in preventing or treating a viral infection mediated by a virus comprising one or more viral RNAs that are translated by a ribosomal shunting mechanism or other non-IRES mediated mechanism. The methods comprise identifying a subject with or at risk of developing a viral infection mediated by a virus comprising one or more viral RNAs that are translated by a ribosomal shunting mechanism or other non-IRES mediated mechanism and administering to the subject a therapeutically effective amount of an agent that reduces ribosomal protein S25 (Rps25) expression or function.

Also provided are methods of inhibiting ribosomal shunting-mediated translation or other non-IRES mediated mechanism. Specifically provided is a method comprising providing a cell, wherein the cell comprises an RNA molecule that is translated by a ribosomal shunting mechanism or other non-IRES mediated mechanism and contacting the cell with an agent that reduces Rps25 expression or function. Reduction of Rps25 expression or function as compared to a control, for example, indicates that the agent inhibits ribosomal shunting-mediated translation or other non-IRES mediated mechanism.

Also provided are methods of screening for an agent that inhibits or promotes ribosomal shunting-mediated translation or other non-IRES mediated translation. Specifically provided is a method comprising providing a system that includes a Rps25 or a nucleic acid that encodes Rps25 and an RNA molecule translated by a ribosomal shunting mechanism or other non-IRES mediated mechanism, contacting the system with the agent to be tested, and determining Rps25 expression or function. A decrease in the level of Rps25 expression or function indicates the agent inhibits ribosomal shunting-mediated translation or other non-IRES mediated translation. An increase in the level of Rps25 expression or function indicates the agent promotes ribosomal shunting-mediated translation or other non-IRES mediated translation.

Also provided are methods of identifying RNA molecules translated by a ribosomal shunting mechanism or other non-IRES mediated mechanism. The methods comprise inhibiting Rps25 expression or function in a cell, determining a protein expression pattern in the cell, and comparing the protein expression pattern in the cell to a control. A decrease in protein expression of a RNA molecule as compared to a control indicates the RNA molecule is translated by a ribosomal shunting mechanism or other non-IRES mediated mechanism.

Further provided are methods of promoting ribosomal shunting-mediated translation or other non-IRES mediated translation. The method comprises providing a cell, wherein the cell comprises an RNA molecule translated by a ribosomal shunting mechanism or other non-IRES mediated mechanism, and contacting the cell with an agent, wherein the agent increases Rps25 expression or activity in comparison to a control. The method can further comprise determining that ribosomal shunting-mediated translation or other non-IRES mediated translation is promoted by detecting an increased level of protein expressed by the RNA molecule translated by a ribosomal shunting mechanism or other non-IRES mediated mechanism in comparison to a control.

The details of one or more embodiments are set forth in the accompanying drawings and the description below. Other features, objects, and advantages will be apparent from the description and drawings, and from the claims.

### DESCRIPTION OF DRAWINGS

FIG. 1 shows a stable knockdown of RPS25 in HeLa cells is viable and the cells do not have significant defects in growth or global translation. FIG. 1A shows light (left) and fluorescence (right) microscopy images of HeLa^{shV} and HeLa^{shS25} cells at 100× magnification. FIG. 1B shows images of a Northern (left) and quantitative western (right) analysis of HeLa^{shV} and HeLa^{shS25} cells. FIG. 1C shows the results of a proliferation assay for HeLa^{shV} and HeLa^{shS25} in 1% or 10% serum. Standard error for n=3 is shown. FIG. 1D shows the protein synthesis rate in 10% serum determined by ³⁵S-methionine incorporation into HeLa^{shV} and HeLa^{shS25} cells. The CPMs were measured for HeLa^{shV} and HeLa^{shS25} and expressed relative to the HeLa^{shV} cells at 100%. Standard error for n=4 is shown.

FIG. 2 shows that HeLa^{shS25} cells exhibit a specific defect in IRES-mediated translation, which can be complemented by exogenous expression of the shRNA resistant RPS25, hS25. FIG. 2A shows a schematic representation of the dual luciferase reporter and the hS25 rescue construct. Transcription of the bicisictronic dual luciferase reporter is controlled by the SV40 or CMV promoter. Cap-dependent translation drives expression of the Renilla luciferase, while the firefly luciferase is under the control of the IGR IRES located in the intercistronic region. The hS25 rescue plasmid is transcribed by the CMV promoter. A gray box indicates the RPS25 shRNA target with the sequence shown below. Arrows point to the synonymous mutations engineered to confer resistance to the shRNA. FIG. 2B shows a graph demonstrating the CrPV IGR IRES activity in cells co-transfected with the dual luciferase reporter, monocistronic cap-dependent β-galactosidase reporter and either the hS25 plasmid or empty pcDNA3 vector. IRES activity (firefly luciferase) was normalized to the cap-dependent translation of β-galactosidase and expressed as a percentage of the activity in HeLa^{shV} cells with the empty vector (black), HeLa^{shS25} with empty vector (dark gray), HeLa^{shV} with hS25 (light gray), HeLa^{shS25} with hS25 (white). Standard error for n=3 is shown. FIG. 2C shows an image of a RPS25 western analysis of cells 24, 48 and 72 hours following transfection with the hS25 rescue plasmid.

FIG. 3 shows viral IRESs that are structurally and functionally different rely on RPS25. FIG. 3A shows a graph demonstrating the normalized activity of several viral IRESs in HeLa^{shS25} cells expressed as a percentage of the activity for each IRES in the control cells. CrPV, cricket paralysis virus intergenic region IRES; HCV, hepatitis C virus; CSFV, classic swine fever virus; EMCV, encephalomyocarditis virus; PV, poliovirus; EV71, enterovirus 71. FIG. 3B shows representative poliovirus plaque assay images and quantification of titers following one round of infection in HeLa^{shV} or HeLa^{shS25} cells. FIG. 3C shows an image of a northern analysis of RPS25 mRNA level in Huh7.13 cells 72 hours after siRNA knockdown. The level of RPS25 mRNA was normalized to the level of β-actin mRNA and expressed as a percentage of the control. FIG. 3D shows the results of replication efficiency of HCV (JHF1 strain) assessed by a quantitative western analysis for the HCV NS5A protein normalized to β-actin 72 hours post infection of control and RPS25 knockdown Huh7.13 cells. FIG. 3E shows representative herpes simplex virus 1 plaque assay images and quantification of viral titers following one round of infection in HeLa^{shV} or HeLa^{shS25} cells. All assays performed in triplicate. Standard error for n=3 is shown.

FIG. 4 shows that RPS25 aids in the translation of cellular IRESs. FIG. 4A shows a graph of cellular IRES activity measured 48 hours after transfection with the bicistronic reporter alone (gray bars) or with hS25 rescue plasmid (white bars) and expressed as a percentage of the activity in the control cells for each IRES (solid line). FIG. 4B shows a polysome analysis of the HeLa^{shV} and HeLa^{shS25} cells. P/M=polysome to monosome ratio. FIG. 4C shows an image of RNA isolated from the HeLa^{shV} and HeLa^{shS25} polysome fractions separated on a denaturing agarose gel. 18S and 28S rRNA are indicated on the ethidium bromide stained gel. The RNA was probed by Northern analysis for p53 and β-actin mRNAs.

FIG. 5 shows that RPS25 is required for ribosomal shunting during adenovirus infection. FIG. 5A shows a diagram of the Ad-hp-Luc adenovirus shunting reporter. A stable stem loop at the 3′ end of the tripartite shunting sequence inhibits normal scanning of the 40S ribosome allowing only shunting to proceed as indicated by the arrow. FIG. 5B shows a graph demonstrating the relative shunting rate determined in HeLa^{shV} (black bars) and HeLa^{shS25} (grey bars) cells co-transfected with the Ad-hp-Luc shunting reporter and β-galactosidase reporter as a control for cap-dependent translation. After one round of Ad5 infection, ribosomal shunting activity (firefly luciferase) was normalized to the β-galactosidase activity and expressed as a percentage of the shunting activity in the mock infected HeLa^{shV} cells. FIG. 5C shows representative plaque assay images and titers following one round of infection with Ad5 adenovirus in HeLa cells. Three independent replicates of each assay were performed and error bars indicate standard error for n=3.

FIG. 6 shows a model for how RPS25 could play a common role in initiation by IRESs and ribosomal shunts but may not be required for cap-dependent initiation. A diagram illustrating a common role for RPS25 to induce a conformational change to open the mRNA binding channel for various mechanisms of initiation. 40S ribosomal subunit (light gray), RPS25 (dark grey). IRES (indicated by arrow), coding region (black), latch for the mRNA tunnel (represented by a black dumbbell closed (unbroken) open (broken)). Some IRESs, such as the CrPV IGR IRES, depend on RPS25 for binding as well as for a conformational change in the 40S subunit in order for the mRNA to be loaded into the binding channel of the 40S subunit. Other IRESs such as HCV bind to the 40S subunit independently of RPS25 and only require it for the conformational change. Ribosomal shunts use a cap-dependent mechanism to initially recruit the 40S subunit to the mRNA, however following transfer of the ribosome from the donor to the acceptor site RPS25 is required to open the mRNA channel for re-loading of the mRNA into the mRNA binding channel. Some IRESs are known to use eIF1 (oval) and eIF1A (circle) and therefore could be able to use these factors to open the mRNA latch if they are present, but could also be able to use RPS25 to trigger opening of the latch in their absence. Cap-dependent translation relies solely on eIF1 and eIF1A to induce the conformational change in the 40S subunit and is not dependent on RPS25 at all.

FIG. 7 shows that a knockdown of RPS25 impaired Dengue Virus (DENV) and Yellow Fever Virus (YFV) replication. FIG. 7A shows a graph demonstrating that knockdown of RPS25 impaired DENV replication. FIG. 7B shows a graph demonstrating that knockdown of RPS25 impaired YFV replication. FIG. 7C shows a graph demonstrating that knockdown of RPS25 did not impair Herpes Simplex Virus-1 (HSV-1) replication.

### DETAILED DESCRIPTION

Provided herein are agents and methods for the treatment or prevention of viral infection or cancer in a subject. The viral infection or cancer is mediated by a virus comprising one or more viral RNAs or cell comprising one or more RNAs that are translated by a ribosomal shunting mechanism or other non-IRES mediated mechanism. The agents and methods comprise reducing ribosomal protein S25 (Rps25) expression or function in the subject.

Optionally, the agents comprise compounds as described below. The compounds for the treatment of viral infections (e.g., cauliflower mosaic virus (CaMV) and adenovirus) include compounds represented by Formula I:

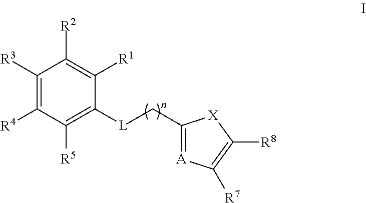

and pharmaceutically acceptable salts or prodrugs thereof.

In Formula I, A is —CR⁹— or —N—. In some examples, A is —CH— or —N—.

Also, in Formula I, L is —O—CR¹⁰R¹¹C(O)—NR⁶—, —NR¹²—NR⁶—, —C(O)—NR⁶—, —SO₂—NR⁶—, —CH₂—NR⁶—, —CH₂—CH₂—NR⁶—, or a substituted or unsubstituted heteroaryl. In some examples, L is a substituted or unsubstituted pyrazole.

Additionally, in Formula I, n is 0, 1, or 2.

Also, in Formula I, X is —CR¹³═CR¹⁴—, —N═CR¹⁵—, —CR¹⁵═N—, —NR¹⁶—, —O—, or —S—. X can be an atom in a five-membered ring or a six-membered ring. For example, when X is —NR¹⁶—, —O—, or —S—, X is an atom of a five-membered ring (e.g., thiophenyl, pyrrolyl, furanyl, oxazolyl, thiazolyl, or imidazolyl). When X is —CR¹³═CR¹⁴—, —N═CR¹⁵—, or —CR¹⁵═N—, X is an atom of a six-membered ring, such as, for example, phenyl, pyridinyl, or pyrazinyl. In some examples, X is —S— or —CH═CH—.

Further, in Formula I, R¹, R², R³, R⁴, R⁵, R⁷, R⁸, R⁹, R¹⁰, R¹¹, R¹³, R¹⁴, and R¹⁵ are each independently selected from hydrogen, halogen, hydroxyl, trifluoromethyl, substituted or unsubstituted thio, substituted or unsubstituted alkoxyl, substituted or unsubstituted aryloxyl, substituted or unsubstituted amino, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl. Optionally. R¹ is hydrogen or methoxy. Optionally, R² is hydrogen, methoxy, or hydroxyl. Optionally, R³ is hydrogen, ethoxy, dimethylamino, methyl, or chloro. Optionally, R⁵ is hydrogen, chloro, methoxy, or hydroxyl. Optionally, R¹⁰ and/or R¹¹ are hydrogen.

Also, in Formula I, R⁶, R¹², and R¹⁶ are each independently selected from hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl, or substituted or unsubstituted carbonyl. Optionally, R⁶, R¹², and/or R¹⁶ are hydrogen.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imidazole, oxazole, pyridine, pyrazole, and pyrazine. Aryl and heteroaryl molecules can also include additional fused rings, for example, benzofuran, indole, benzothiophene, naphthalene, anthracene, and quinoline.

The alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl molecules used herein can be substituted or unsubstituted. As used herein, the term substituted includes the addition of an alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl group to a position attached to the main chain of the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl, e.g., the replacement of a hydrogen by one of these molecules. Examples of substitution groups include, but are not limited to, hydroxyl, halogen (e.g., F. Br, Cl, or I), and carboxyl groups. Conversely, as used herein, the term unsubstituted indicates the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl has a full complement of hydrogens, i.e., commensurate with its saturation level, with no substitutions, e.g., linear decane (—(CH₂)₉—CH₃).

In Compound I, adjacent R groups on the phenyl ring, i.e., R¹, R², R³, R⁴, and R⁵, can be combined to form substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl groups. For example, R⁵ can be a formamide group and R⁶ can be an ethylene group that combine to form a pyridinone group. Other adjacent R groups include the combinations of R¹ and R², R² and R³, and R³ and R⁴.

Specific examples of Formula I are as follows:

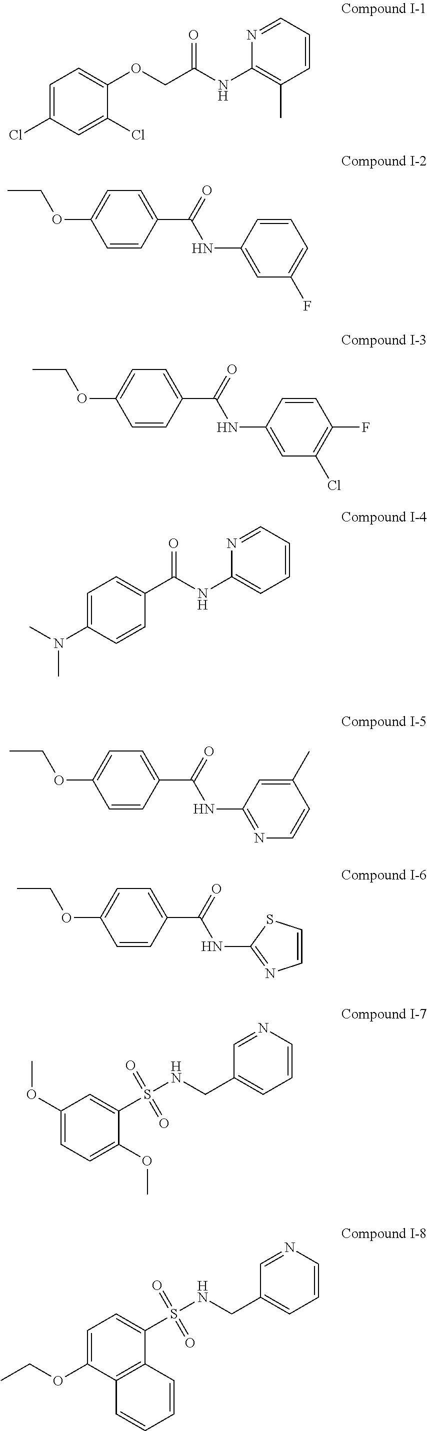

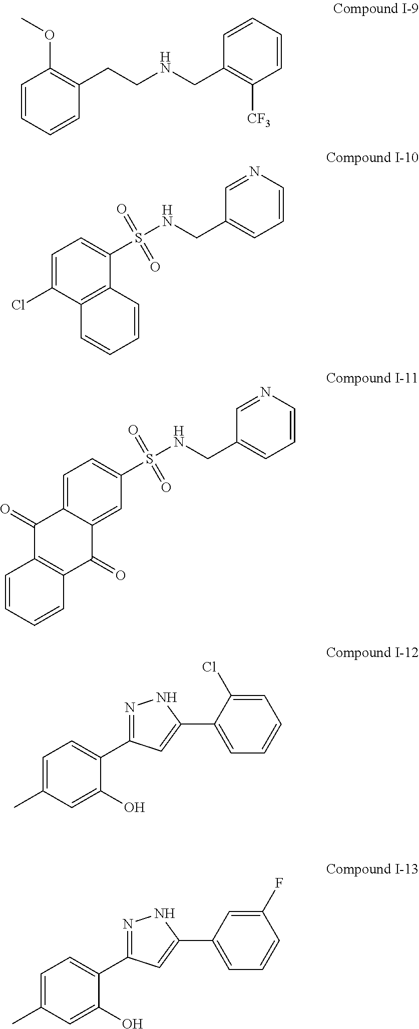 [SMILES: COc1ccccc1CCNCc1ccccc1C(F)(F)F ; O=S(=O)(NCc1cccnc1)c1ccc(Cl)c2ccccc12 ; O=C1c2ccccc2C(=O)c2cc(S(=O)(=O)NCc3cccnc3)ccc21 ; Cc1ccc(-c2cc(-c3ccccc3Cl)[nH]n2)c(O)c1 ; Cc1ccc(-c2cc(-c3cccc(F)c3)[nH]n2)c(O)c1]

Variations on the Formula I include the addition, subtraction, or movement of the various constituents as described for each compound. Similarly, when one or more chiral centers are present in a molecule, the chirality of the molecule can be changed. The compounds described herein can be isolated in pure form or as a mixture of isomers. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The compounds described herein can be prepared in a variety of ways known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

Provided herein are methods of treating or preventing a viral infection in a subject. The methods comprise identifying a subject with or at risk of developing a viral infection, wherein the viral infection is mediated by a virus comprising one or more viral RNAs that are translated by a ribosomal shunting mechanism or other non-IRES mediated mechanism and administering to the subject a therapeutically effective amount of an agent that reduces Rps25 expression or function in the subject in comparison to a control.

As used throughout, the agent that reduces Rps25 expression or function can, for example, be selected from the group consisting of a small molecule, a polypeptide, a nucleic acid molecule, a peptidomimetic, or a combination thereof. Optionally, the nucleic acid molecule is selected from the group consisting of an antisense molecule, a short-interfering RNA (siRNA) molecule, a microRNA (miRNA) molecule, a RNA aptamer, or a combination thereof. The siRNA molecule can, for example, comprise SEQ ID NO:3.

Optionally, the virus is selected from the group consisting of a cauliflower mosaic virus (CaMV), a Sendai paramyxovirus, a rice tungro bacilliform virus, a human papilloma virus (e.g., human papilloma virus type 18), a duck hepatitis B virus (DHBV), a prototype foamy virus, and an adenovirus (e.g., human type C adenovirus). Optionally, the virus is selected from the Flaviviridae family. The flavivirus can, for example, be selected from the group consisting of a dengue fever virus, a yellow fever virus, or a West nile virus.

Also provided are methods of treating or preventing a cancer in a subject. The methods comprise identifying a subject with or at risk of developing a cancer, wherein the cancer is mediated by a one or more RNAs that are translated by a ribosomal shunting mechanism or other non-IRES mediated mechanism, and administering to the subject a therapeutically effective amount of an agent that reduces or increases Rps25 expression or function in the subject. Optionally, the agent reduces Rps25 expression or function in the subject in a cancer related to increased ribosomal shunting-mediated translation or other non-IRES mediated translation. Optionally, the agent increases Rps25 expression or function in the subject in a cancer related to decreased ribosomal shunting-mediated translation or other non-IRES mediated translation.

As defined herein, a cancer related to increase or decreased ribosomal shunting-mediated translation or other non-IRES mediated translation is a cancer cause by, a cancer that metastasizes due to, and/or a cancer present that exhibits an increase or decrease in translation of one or more RNAs by a ribosomal shunting-mediated mechanism or a non-IRES mediated mechanism. The increase or decrease in translation of one or more of the RNAs contributes to any timepoint in the lifespan of the cancer, from the birth of the cancer through the metastasis of the cancer. Examples of cancers include, but are not limited to, breast cancer, prostate cancer, lung cancer, liver cancer, pancreatic cancer, thyroid cancer, skin cancer, testicular cancer, ovarian cancer, mouth/esophageal cancer, and/or brain cancer.

Also provided herein is a method of inhibiting ribosomal shunting-mediated translation or other non-IRES mediated translation. The method comprises providing a cell, wherein the cell comprises an RNA molecule that is translated by a ribosomal shunting mechanism or other non-IRES mediated mechanism and contacting the cell with an agent that reduces Rps25 expression or function. Reduction of Rps25 expression or function as compared to a control indicates the agent inhibits ribosomal shunting-mediated translation or other non-IRES mediated translation. Optionally, the method further comprises determining that ribosomal shunting-mediated translation or other non-IRES mediated translation is inhibited by determining a reduced level of protein expressed by the RNA molecule translated by ribosomal shunting or other non-IRES mediated mechanism in comparison to a control. The expression of Rps25 can be reduced by decreasing the level of Rps25 RNA or protein expression. The function of Rps25 can, for example, be reduced by blocking binding of Rps25 to the 40S subunit of the ribosome.

Optionally, the RNA translated by ribosomal shunting is selected from the group consisting of HSP70, cIAP2, or beta-secretase.

Also provided is a method of screening for an agent that inhibits or promotes ribosomal shunting-mediated translation or other non-IRES mediated translation. The method comprises providing a system comprising a Rps25 or a nucleic acid that encodes Rps25 and an RNA molecule translated by a ribosomal shunting mechanism or other non-IRES mediated mechanism, contacting the system with the agent to be screened, and determining Rps25 expression or function. A decrease in the level of Rps25 expression or function indicates the agent inhibits ribosomal shunting-mediated translation or other non-IRES mediated translation. An increase in the level of Rps25 expression or function indicates the agent promotes ribosomal shunting-mediated translation or other non-IRES mediated translation. Optionally, the system comprises a cell. The cell can contain naturally occurring RNA molecules translated by a ribosomal shunting mechanism or other non-IRES mediated mechanism. The cell can also be modified to contain artificial RNA molecules translated by a ribosomal shunting mechanism or other non-IRES mediated mechanism. Optionally, the system comprises an in vitro assay. The agent to be tested can, for example, be selected from the group consisting of a small molecule, a polypeptide, a nucleic acid molecule, a peptidomimetic, or a combination thereof. Also provided are agents isolated by the methods of screening described herein.

Also provided is a method of identifying RNA molecules translated by a ribosomal shunting mechanism or other non-IRES mediated mechanism. The methods comprise inhibiting Rps25 expression or function in a cell, determining a protein expression pattern in the cell; and comparing the protein expression pattern to a control. A decrease in protein expression of an RNA molecule as compared to a control indicates the RNA molecule is translated by a ribosomal shunting mechanism or other non-IRES mediated mechanism. The methods can comprise identifying a novel RNA molecule that is translated by a ribosomal shunting mechanism or other non-IRES mediated mechanism or verifying a previously hypothesized RNA molecule translated by a ribosomal shunting mechanism or other non-IRES mediated mechanism. Rps25 expression of function can be inhibited using the agents described herein, e.g., the siRNA comprising SEQ ID NO:3. Determining the protein expression pattern of a cell can, for example, comprise doing a protein array or performing a deep sequencing assay on polysomal fractions within the cell. Alternatively, determining the protein expression pattern can comprise using other methods of determining protein expression known in the art.

Further provided is a method of promoting ribosomal shunting-mediated translation or other non-IRES mediated translation, the method comprising providing a cell, wherein the cell comprises an RNA molecule translated by a ribosomal shunting mechanism or other non-IRES mediated mechanism and contacting the cell with an agent that increases Rps25 expression or function in comparison to a control. An increase in Rps25 expression or function indicates the agent promotes ribosomal shunting-mediated translation or other non-IRES mediated translation. Optionally, the method further comprises determining that ribosomal shunting-mediated translation or other non-IRES mediated translation is promoted by detecting an increased level of protein encoded by the RNA molecule translated by a ribosomal shunting mechanism or other non-IRES mediated mechanism in comparison to a control.

By control is meant in the absence of treatment or in the absence of an agent or composition. Thus, a control can be a known standard, or the subject, cell, or system before treating or after the effect of treatment has subsided. A control can also be an untreated subject, cell, or system.

Also provided is a method of promoting ribosomal shunting-mediated translation or other non-IRES mediated translation, the method comprising providing a cell with a nucleic acid encoding a Rps25 protein or a functional fragment thereof. Such a method can be in vivo or in vitro.

As described herein, an RNA molecule translated by a ribosomal shunting mechanism or other non-IRES mediated mechanism can be artificially created or naturally occurring. An artificially created RNA molecule can, for example, be a firefly luciferase mRNA that contains a ribosomal shunting control element or other non-IRES mediated control element that controls translation of the firefly luciferase protein. An artificially created RNA molecule can also be a green fluorescent protein mRNA that contains a ribosomal shunting control element or other non-IRES mediated control element that controls translation of the green fluorescent protein. These RNA molecules are generally used as reporters for ribosomal shunting-mediated translation or other non-IRES mediated translation. A naturally occurring RNA molecule translated by a ribosomal shunting mechanism or non-IRES mediated translation mechanism can, for example, be a cellular or a viral RNA molecule. A cellular RNA translated by a ribosomal shunting mechanism can for example, be selected from the group consisting of HSP70, cIAP2, and beta-secretase. A viral RNA molecule translated by a ribosomal shunting mechanism can be found in a virus selected from the group consisting of a cauliflower mosaic virus (CaMV), a Sendai paramyxovirus, a rice tungro bacilliform virus, a human papilloma virus (e.g., human papilloma virus type 18), a duck hepatitis B virus (DHBV), a prototype foamy virus, and an adenovirus (e.g., human type C adenovirus). A viral RNA molecule translated by a non-IRES mediated mechanism can be found in a virus from the Flaviviridae family (e.g., a dengue virus, a yellow fever virus, or a West Nile virus).

Without intending to be limited by theory, as described herein, translation by a ribosomal shunting mechanism means that the RNA is translated in the following manner. The 40S ribosomal subunit is recruited to the 5′ end of the mRNA through a cap-dependent mechanism, the 40S subunit scans the mRNA in a 5′ to 3′ direction and sometimes translates a short open reading frame, and then the 40S subunit is transferred from a shunt donor region to a shunt acceptor region bypassing, without scanning) regions of mRNA sequence or structure to initiate translation at a downstream AUG of the mRNA. Ribosomal shunting mediated translation can, for example, be observed during times of cellular stress.

The vast majority of mRNAs are translated using a cap-dependent mechanism whereby several translation initiation factors work in concert to bring the 40S subunit to the cap structure at the 5′ end of the mRNA. However, many viruses are able to subvert the host translational machinery by using alternate mechanisms of initiating translation (e.g., IRES-mediated translation). As described herein, translation by a non-IRES mediated mechanism can, for example, encompass translation of an mRNA in a manner that does not rely on cap-dependent mediated translation or an IRES mediated translation mechanism. Viruses, such as dengue virus, yellow fever virus, and West Nile virus, can initiate translation by using a non-IRES mediated mechanism.

As described herein, the level of Rps25 protein expression can, for example, be determined using an assay selected from the group consisting of Western blot, enzyme-linked immunosorbent assay (ELISA), enzyme immunoassay (EIA), radioimmunoassay (RIA), or protein array. The level of Rps25 RNA expression can, for example, be determined using an assay selected from the group consisting of microarray analysis, gene chip, Northern blot, in situ hybridization, reverse transcription-polymerase chain reaction (RT-PCR), one step PCR, and real-time quantitative real time (qRT)-PCR. The analytical techniques to determine protein or RNA expression are known. See, e.g., Sambrook et al., Molecular Cloning: A Laboratory Manual, 3rd Ed., Cold Spring Harbor Press, Cold Spring Harbor, NY (2001).

As described herein, the level of Rps25 function can, for example, be determined by using an assay selected from the group consisting of an RNA mobility shift assay, an RNA crosslinking assay, an RNA affinity assay, a protein-protein binding assay, and an assay measuring ribosomal shunting-mediated translation of an RNA molecule translated by a ribosomal shunting mechanism. A decrease in Rps25 function can, for example, be demonstrated by a loss of binding to an RNA molecule translated by a ribosomal shunting mechanism, a loss of binding to the 40S ribosomal subunit, or a decrease in ribosomal shunting-mediated translation of an RNA molecule translated by a ribosomal shunting mechanism as compared to a control. An increase in Rps25 function can, for example, be demonstrated by an enhanced binding to an RNA molecule translated by a ribosomal shunting mechanism, an enhanced binding to the 40S ribosomal subunit, or an increase in ribosomal shunting-mediated translation of an RNA molecule translated by a ribosomal shunting mechanism as compared to a control.

As used herein an agent can, for example, be selected from the group consisting of a small molecule, a polypeptide, a nucleic acid molecule, a peptidomimetic, or a combination thereof. Optionally, the polypeptide is an antibody (e.g., to Rps25, to the 40S ribosomal subunit). Optionally, the nucleic acid molecule is an Rps25 inhibitory nucleic acid molecule.

An Rps25 inhibitory nucleic acid molecule can, for example, be selected from the group consisting of an antisense molecule, a short-interfering RNA (siRNA) molecule, a microRNA (miRNA) molecule, a RNA aptamer, or a combination thereof.

A 21-25 nucleotide siRNA or miRNA sequence can, for example, be produced from an expression vector by transcription of a short-hairpin RNA (shRNA) sequence, a 60-80 nucleotide precursor sequence, which is subsequently processed by the cellular RNAi machinery to produce either an siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.). Qiagen (Valencia, Calif.), and Ambion (Austin, Tex.). A siRNA sequence preferably binds a unique sequence within the Rps25 mRNA with exact complementarity and results in the degradation of the Rps25 mRNA molecule. A siRNA sequence can bind anywhere within the Rps25 mRNA molecule. Optionally, the Rps25 siRNA sequence can target the sequence 5′-GGACUUAUCAAACUGGUUU-3′ (SEQ ID NO:5), corresponding to nucleotides 283-301 of the human Rps25 mRNA nucleotide sequence, wherein position 1 begins with the first nucleotide of the coding sequence of the Rps25 mRNA molecule at Accession Number NM_{—}001028 on GenBank. Optionally, the siRNA sequence comprises SEQ ID NO:3. A miRNA sequence preferably binds a unique sequence within the Rps25 mRNA with exact or less than exact complementarity and results in the translational repression of the Rps25 mRNA molecule. A miRNA sequence can bind anywhere within the Rps25 mRNA sequence, but preferably binds within the 3′ untranslated region of the Rps25 mRNA molecule. Methods of designing siRNA and miRNA molecules are known in the art, see, e.g., Peek and Behlke, Curr. Opin. Mol. Ther. 9(2):110-8 (2007); Patzel, Drug Discov. Today 12(3-4):139-48 (2007); Takasaki, Methods Mol. Biol. 487:1-39 (2009); Aronin, Gene Ther. 13(6):509-16 (2006); Sablok et al., Biochem. Biophys. Res. Commun. 406(3):315-9 (2011); Wang, Methods Mol. Biol. 676:211-23 (2011); Tilesi et al., Curr. Opin. Mol. Ther. 11(2):156-64 (2009). Methods of delivering siRNA or miRNA molecules are known in the art, e.g., see Oh and Park, Adv. Drug. Deliv. Rev. 61(10):850-62 (2009); Gondi and Rao, J. Cell Physiol. 220(2):285-91 (2009); and Whitehead et al., Nat. Rev. Drug Discov. 8(2): 129-38 (2009).

Antisense nucleic acid sequences can, for example, be transcribed from an expression vector to produce an RNA which is complementary to at least a unique portion of the Rps25 mRNA and/or the endogenous gene which encodes Rps25. Hybridization of an antisense nucleic acid under specific cellular conditions results in inhibition of Rps25 protein expression by inhibiting transcription and/or translation.

Antibodies described herein bind the Rps25 and antagonize the function of the Rps25. Optionally, the antibodies described herein bind Rps25 and inhibit the binding of Rps25 to the 40S subunit of the ribosome. The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

As used herein, the term antibody encompasses, but is not limited to, whole immunoglobulin (i.e., an intact antibody) of any class. The term antibody or fragments thereof can also encompass chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies that maintain Rps25 binding activity are included within the meaning of the term antibody or fragment thereof.

Optionally, the antibody is a monoclonal antibody. The term monoclonal antibody as used herein refers to an antibody from a substantially homogeneous population of antibodies, i.e., the individual antibodies comprising the population are identical except for possible naturally occurring mutations that may be present in minor amounts. Monoclonal antibodies may be prepared using hybridoma methods, such as those described by Kohler and Milstein, Nature, 256:495 (1975) or Harlow and Lane, Antibodies, A Laboratory Manual. Cold Spring Harbor Publications, New York (1988). The monoclonal antibodies may also be made by recombinant DNA methods, such as those described in U.S. Pat. No. 4,816,567.

Optionally, Rps25 is human. Optionally, Rps25 is non-human (e.g., rodent, canine, feline, insect, or plant). There are a variety of sequences that are disclosed on Genbank, and these sequences and others are herein incorporated by reference in their entireties as are individual subsequences or fragments contained therein. As used herein, Rps25 refers to the ribosomal S25 polypeptide and homologs, variants, and isoforms thereof. For example, the nucleotide and amino acid sequences of human Rps25 be found at GenBank Accession Nos. NM_{—}001028 and NP_{—}001019.1, respectively. Thus provided is the nucleotide sequence of Rps25 comprising a nucleotide sequence at least about 70%, 75%, 80%, 85%, 90%, 95%, 98%, 99% or more identical to the nucleotide sequence of the aforementioned GenBank Accession Number. Also provided is the amino acid sequence of Rps25 comprising an amino acid sequence at least about 70%, 75%, 80%, 85%, 90%, 95%, 98%, 99% or more identical to the sequence of the aforementioned GenBank Accession Number.

Nucleic acids that encode the polypeptide sequences, variants, and fragments thereof are disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequences.

As used herein, the term peptide, polypeptide or protein is used to mean a molecule comprised of two or more amino acids linked by a peptide bond. Protein, peptide, and polypeptide are also used herein interchangeably to refer to amino acid sequences. It should be recognized that the term polypeptide or protein is not used herein to suggest a particular size or number of amino acids comprising the molecule and that a polypeptide of the disclosure can contain up to several amino acid residues or more.

As with all peptides, polypeptides, and proteins, including fragments thereof, it is understood that additional modifications in the amino acid sequence of the variant Rps25 polypeptides can occur that do not alter the nature or function of the peptides, polypeptides, or proteins. Such modifications include conservative amino acids substitutions and are discussed in greater detail below.

The polypeptides provided herein have a desired function. Rps25 is part of a ribosomal complex that promotes ribosomal shunting-mediated translation. The polypeptides are tested for their desired activity using the in vitro assays described herein.

The polypeptides described herein can be further modified and varied so long as the desired function is maintained. It is understood that one way to define any known modifications and derivatives or those that might arise, of the disclosed genes and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to Rps25 and variants provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol. Biol. 48:443 (1970), by the search for similarity method of Pearson and Lipman. Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989); Jaeger et al., Proc. Natl. Acad. Sci. USA 86:7706-10 (1989); Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations (e.g., due to genetic polymorphism), may arise due to environmental influence (e.g., by exposure to ultraviolet light), or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. Amino acid substitutions are typically of single residues, but can occur at a number of different locations at once: insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at least one residue has been removed and a different residues inserted in its place. Such substitutions generally are made in accordance with the following Table 1 and are referred to as conservative substitutions.

TABLE 1
Amino Acid Substitutions

| Amino Acid | Substitutions (others are known in the art) |
|---|---|
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu,Ile, Val |
| Phe | Mel, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Modifications, including the specific amino acid substitutions, are made by known methods. By way of example, modifications are made by site-specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

Provided herein are methods of treating or preventing viral infection or cancer in a subject. Such methods include administering an effective amount of the compounds disclosed herein or an agent comprising a small molecule, a polypeptide, a nucleic acid molecule, a peptidomimetic or a combination thereof. Optionally, the small molecules, polypeptides, nucleic acid molecules, and/or peptidomimetics are contained within a pharmaceutical composition.

Provided herein are compositions containing the provided small molecules, polypeptides, nucleic acid molecules, and/or peptidomimetics, optimally with a pharmaceutically acceptable carrier described herein. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy. Optionally, the composition is administered by oral inhalation, nasal inhalation, or intranasal mucosal administration. Administration of the compositions by inhalant can be through the nose or mouth via delivery by spraying or droplet mechanism. For example, in the form of an aerosol. In the case of cancer treatment, the composition or agent can be administered directly into or onto a tumor.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Poliovirus, Sindbis, and other RNA viruses, including these viruses with the HIV backbone. Also preferred are any viral families which share the properties of these viruses, which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example. Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

As used throughout, subject can be a vertebrate, more specifically a mammal (e.g. a human, horse, cat, dog, cow, pig, sheep, goat, mouse, rabbit, rat, and guinea pig), birds, reptiles, amphibians, fish, and any other animal. A subject can, for example, also be a plant or insect that is capable of being infected by a virus that comprises one or more viral RNAs translated by a ribosomal shunting mediated mechanism or a non-IRES mediated mechanism. The term does not denote a particular age or sex. Thus, adult and newborn subjects, whether male or female, are intended to be covered. As used herein, patient or subject may be used interchangeably and can refer to a subject with a disease or disorder (e.g. viral infection or cancer). The term patient or subject includes human, veterinary subjects, plants, and/or insects.

Subjects include those with or at risk of developing cancer or with or at risk of viral infection. A subject at risk of developing cancer can be genetically predisposed to the cancer, e.g., have a family history or have a mutation in a gene that causes the cancer or may be immunocompromised. A subject at risk of developing a viral infection can be predisposed to the viral infection, e.g., have an occupation putting the subject at risk for contracting a viral infection, have a compromised immune system, or have been exposed to a virus. A subject currently with a cancer or viral infection has one or more than one symptoms of the cancer or viral infection and may have been diagnosed with the cancer or viral infection.

The methods and agents as described herein are useful for both prophylactic and therapeutic treatment. For prophylactic use, a therapeutically effective amount of the agent described herein is administered to a subject prior to onset (e.g., before obvious signs of cancer or a viral infection) or during early onset (e.g., upon initial signs and symptoms of cancer or a viral infection). Prophylactic administration can occur for several days to years prior to the manifestation of symptoms of a viral infection. Prophylactic administration can be used, for example, in the preventative treatment of subjects occupationally exposed to viruses or in subjects diagnosed with a genetic predisposition to cancer. Therapeutic treatment involves administering to a subject a therapeutically effective amount of the agents described herein after diagnosis or development of cancer or a viral infection.

According to the methods taught herein, the subject is administered an effective amount of the agent. The terms effective amount and effective dosage are used interchangeably. The term effective amount is defined as any amount necessary to produce a desired physiologic response (e.g., a decrease in the level of ribosomal shunting-mediated translation or non-IRES mediated translation resulting in the treatment of cancer or a viral infection). Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease (e.g., cancer) or condition (e.g., viral infection) or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. Treatment can also include a delay in the progression of one or more symptoms. It is understood that treatment does not necessarily refer to a cure or complete ablation of the disease, condition, or symptoms of the disease or condition. Thus, treatment refers, for example, to an improvement in one or more symptoms of a viral infection or a cancer.

As used herein, the terms prevent, preventing, and prevention of a disease (e.g., cancer) or condition (e.g., viral infection) refers to an action, for example, administration of a therapeutic agent, that occurs before or at about the same time a subject begins to show one or more symptoms of the disease or condition, which inhibits or delays onset or exacerbation of one or more symptoms of the disease or condition. As used herein, references to decreasing, reducing, or inhibiting include a change of 10%, 20%, 30%, 40%, 50%, 60%, 70%, 800/%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Materials and Methods

Cell Culture.

HeLa cells (Invitrogen; Carlsbad, Calif.) were cultured in complete media (high glucose Dulbecco's modified Eagle's medium [DMEM] supplemented with 10% [v/v] fetal bovine serum, 1% [v/v]L-glutamine and 1% [v/v] penicillin/streptomycin). Huh7.5 cells were cultured in complete media supplemented with 1% nonessential amino acids. All cells were maintained at 37° C. and 5% CO₂.

Cloning.

To generate pLVTHMshS25 shRNA the pLVTHM vector (Addgene plasmid 12247) (Addgene; Cambridge, Mass.) was digested with ClaI and MluI. The RPS25 shRNA insert was generated by annealing and phosphorylating (T4 Kinase) (Promega; Madison, Wis.) the complementary DNA oligos: (sense, 5′-cgcgtccccGGACTT ATCAAACTGGTTTttcaagagaAAACCAGTTTGATAAGTCCtttttggaaat-3′ (SEQ ID NO: 1) and antisense, 5′-cgatttccaaaaaCCTGAATAGTTTGACCAAAagagaactt TTTGGTCAAACTATTCAGGccct-3′ (SEQ ID NO:2)) (IDT; Coralville, Iowa). The shRNA insert was inserted into the restricted pLVTHM vector and verified by sequencing.

The CrPV, HCV, EMCV, PV and EV71 plasmids have been previously described (Landry et al., Genes Dev. 23:2753-64 (2009): Thompson et al., Proc. Natl. Acad. Sci. USA 98:12972-7 (2001)). To construct the CSFV IRES dual luciferase reporter the CSFV IRES plus 69 bases of the coding region was amplified from the pXLcsfv1-442 plasmid using primer set P1 (see table 2) (Fletcher and Jackson, J. Virol. 76:5024-33 (2002)) and cloned into the EcoRI/NcoI digested pΔEMCV plasmid (Carter and Sarnow, J. Biol. Chem. 275:28301-7 (2000)). The p53 IRES (nucleotides 64-197 [numbering based on reference sequence NM_{—}000546.4]) was amplified from HeLa cDNA using primer set P2 and cloned into the EcoRI/NcoI restricted pΔEMCV plasmid. To verify that only the full length dual luciferase transcript was produced by the pΔEMCV-CSFV and pΔEMCV-p53 reporter plasmids, a northern analysis against the firefly luciferase gene was performed on poly(A) isolated RNA from HeLa cells transfected with the indicated reporter plasmid. A single product ensures that firefly luciferase activity originated from IRES-mediated translation. The Apaf-1, Bag-1, c-myc, KMI2, L-myc, MNT, MTG8a, myb and Set7 cellular IRESs are pRF dual luciferase plasmids and have been described and verified elsewhere (Stoneley et al., Oncogene 16:423-8 (1998); Coldwell et al., Oncogene 19:899-905 (2000); Coldwell et al., Oncogene 20:4095-100 (2001); Jopling et al., RNA 10:287-98 (2004); Mitchell et al., Genes Dev. 19:1556-71 (2005); Bushell et al., Mol. Cell. 23:401-12 (2006)).

TABLE 2
Primer Sequences (restriction enzymes are in lowercase)

| Primer Set | | Sense (5′ to 3′) | Antisense (5′ to 3′) |
|---|---|---|---|
| CSFV | P1 | gaattcGTCGACGAGGTTAGCTCTTTC | ccatggTACCCGGTTCCTC |
| | | (SEQ ID NO: 5) | (SEQ ID NO: 25) |
| p53 | P2 | gaattcTCTAGAGCCACCGTCCAGG | ccatggGGCAGTGACCCGGAAGGCAG |
| | | (SEQ ID NO: 6) | (SEQ ID NO: 26) |
| hS25 | P3 | GCTAGCATGCCGCCTAAGGACGACAAGA | |
| | | AGAAGAAGG | |
| | | (SEQ ID NO: 7) | |
| | | TTGTCTTTCTTGGCCGACTTTCCAGCGTCC | |
| | | TTCTTCTTCTTGTCGTCC | |
| | | (SEQ ID NO: 8) | |
| | | GAAAGTCGGCCAAGAAAGACAAAGACCC | |
| | | AGTGAACAAATCCGGGGGC | |
| | | (SEQ ID NO: 9) | |
| | | GCCTTTGGACCACTTCTTCTTTTTGGCCTT | |
| | | GCCCCCGGATTTGTTCA | |
| | | (SEQ ID NO: 10) | |
| | | AAAAGAAGAAGTGGTCCAAAGGCAAAGT | |
| | | TCGGGACAAGCTCAATAACTTAG | |
| | | (SEQ ID NO: 11) | |
| | | TTATCATAGGTAGCTTTGTCAAACAAGAC | |
| | | TAAGTTATTGAGCTTGTCCCGAAC | |
| | | (SEQ ID NO: 12) | |
| | | TCTTGTTTGACAAAGCTACCTATGATAAA | |
| | | CTCTGTAAGGAAGTTCCCAACTAT | |
| | | (SEQ ID NO: 13) | |
| | | GAGACCACAGCTGGGGTTATAAGTTTATA | |
| | | GTTGGGAACTTCCTTACAGAG | |
| | | (SEQ ID NO: 14) | |
| | | TATAACCCCAGCTGTGGTCTCTGAGAGAC | |
| | | TGAAGATECGAGGCTCCC | |
| | | (SEQ ID NO: 15) | |
| | | GAGCTCCTGAAGGGCTGCCCTGGCCAGGG | |
| | | AGCCTCGAATCTTCAG | |
| | | (SEQ ID NO: 16) | |
| | | CAGCCCTTCAGGAGCTCCTTAGTAAAGGC | |
| | | CTGATTAAGCTCGTG | |
| | | (SEQ ID NO: 17) | |
| | | GGTGTAAATTACTTGAGCTCTGTGCTTTG | |
| | | ACACGAGCTTAATCAGGCC | |
| | | (SEQ ID NO: 18) | |
| | | CACAGAGCTCAAGTAATTTACACCAGAAA | |
| | | TACCAAGGGTGGAGATGCTC | |
| | | (SEQ ID NO: 19) | |
| | | GGATCCTCATGCATCTTCACCAGCAGGIG | |
| | | GAGCATCTCCACCCTTGGT | |
| | | (SEQ ID NO: 20) | |
| hS25 | P4 | gctagcATGCCGCCTAA | ggatccTCATGCATCTTCACC |
| flank | | (SEQ ID NO: 21) | (SEQ ID NO: 27) |
| RPS25 | P5 | ATGCCGCCTAAGGACGAC | TCATGCATCTTCACCAGC |
| | | (SEQ ID NO: 22) | (SEQ ID NO: 28) |
| Actin | P6 | GCACTCTTCCAGCCTTCC | GCGCTCAGGAGGGAGCAAT |
| | | (SEQ ID NO: 23) | (SEQ ID NO: 29) |
| shRPS25 | P7 | cgcgtccccGGACTTATCAAACTGGTTTttcaagag | cgatttccaaaaaCCTGAATAGTTTGA |
| | | aAAACCAGTTTGATAAGTCCtttttggaaa | CCAAAagagaacttTTTGGTCAAAC |
| | | (SEQ ID NO: 24) | TATTCAGGcccct |
| | | | (SEQ ID NO: 30) |

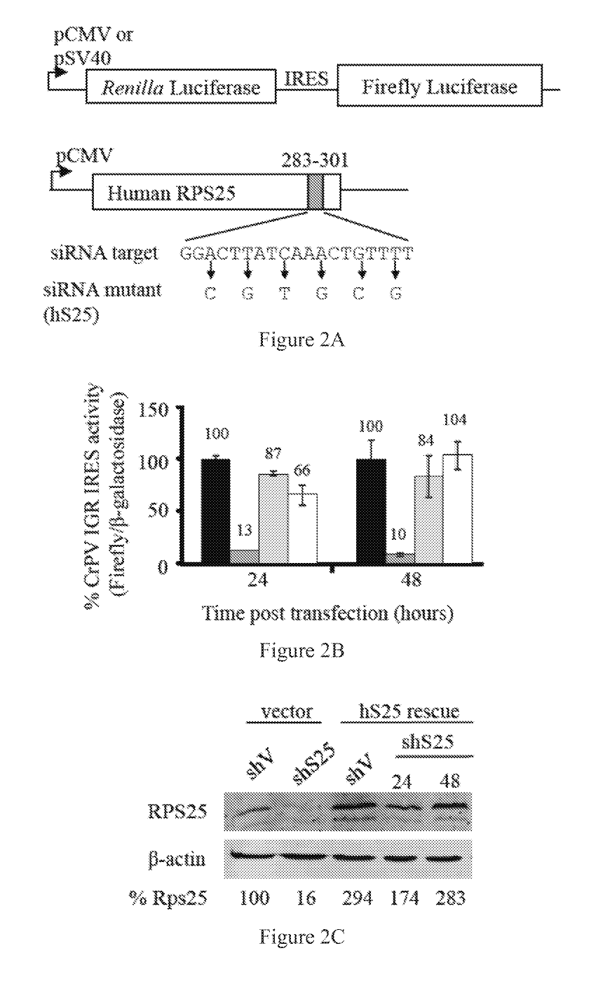

To create the hS25 rescue plasmid, the RPS25 coding region (bases 64-441 based on reference sequence NM_{—}0001028) modified with six synonymous mutations in the siRNA recognition site (nucleotides 283-301, FIG. 2A) was synthesized by long oligo PCR and cloned into the NheI and BamHI sites of the dual luciferase plasmid pSRT222 plasmid replacing the entire dual luciferase cassette with the RPS25 coding region (Landry et al., Genes Dev. 23:2753-64 (2009)). Long oligonucleotides were designed using the assembly PCR oligomaker and long oligo PCR was carried out as described previously (Rydzanicz et al., Nucleic Acids Res. 33:W521-5 (2005)). Briefly. A PCR reaction with primer set P3 was used to assemble the long oligomers of DNA (one cycle at 94° C. for 4 minutes; then 8 cycles of 94° C. for 60 seconds, 54° C. for 2 minutes, 72° C. for 3 minutes; followed by a final single cycle at 72° C. for 5 minutes). A 2 μl aliquot of this reaction was added to the second stage PCR reaction with 20-mer flanking primers (primer set P4) encoding NheI and BamHI sites on their termini to facilitate cloning into pSRT222 (that was denatured for 94° C. for 5 minutes; then 24 cycles of 94° C. for 30 seconds. 54° C. for 2 minutes, 72° C. for 90 seconds; followed by a final extension cycle at 72° C. 5 minutes). All cloning was verified by sequencing.

Lentiviral Vectors.

Virus was generated by co-transfection of pLVTHMshS25, psPAX2 packaging plasmid (Addgene; plasmid 12260) and pMG2.G, a VSV-G envelope plasmid (Addgene; plasmid 12259) into HEK293T cells. After 24 hours, supernatant was collected every 12 hours for 2 days. The viral supernatant was filter sterilized using a 0.2 μm filter and applied directly to the HeLa cells.

Proliferation Assay.

3×10⁴ cells were seeded into 6-well plates and media was replaced with either 1% or 10% serum after 24 hours. Viable cells were counted at 1, 2, 3 and 4 days by removing them from the plate with trypsin and staining with trypan blue and manually counting the cells using a hemocytometer. The cells were fed with the indicated media every 24 hours. Each assay was performed in triplicate.

Global Protein Synthesis Rate.

To pulse label cells, 1×10⁵ HeLa^{shS25} or HeLa^{shV} vector control cells were grown to 70% confluency in 12-well plates and then were incubated in DMEM media supplemented with dialyzed FBS lacking methionine and cysteine to starve the cells of the sulfur containing amino acids for 15 minutes. The cells were radiolabeled for 20 minutes in the same media supplemented with 0.1 mCi ³⁵S protein labeling mix (PerkinElmer; Waltham, Mass.). TCA precipitation was performed as described previously (Landry et al., Genes Dev. 23:2753-64 (2009)). Briefly, cells were lysed with E1 lysis buffer (50 mM HEPES pH 7.0, 250 mM NaCl, 0.1% NP-40) for 30 minutes on ice. 20 μl of the lysate was mixed with 100 μl BSA/NaN₃ (1 mg/ml BSA, 0.2% NaN₃) and 1 ml of 10% TCA to precipitate the proteins. Precipitates were filtered over a glass microfiber filter, washed with 10% TCA, followed by 100% ethanol. The radioactivity of the precipitates on the filter was measured with a Wallac 1409 liquid scintillation counter (PerkinElmer; Waltham, Mass.).

Transfections.

The day before transfection, 5×10⁴ HeLa cells were seeded into 24-well plates. Once the cells reached 90% confluency, transfection of dual luciferase reporters was done using Lipofectamine 2000 (Invitrogen) according to the manufacturer's recommendations using 0.4 μg DNA per well. Cells were harvested for luciferase analysis after 24 hours. Double stranded RPS25 siRNAs, 5′-GGACUUAUCAAACUGGUUUtt-3′ (SEQ ID NO:3) and 5′-AAACCAGUUUGAUAAGUCCtt-3′ (SEQ ID NO:4) (Ambion, siRNA ID #142220), were used to knock down RPS25 in Huh7 cells. The Dicector™ DS (IDT; Coralville, Iowa) scrambled negative control duplex was used as a negative control. siRNA complexes were prepared in opti-MEM (Invitrogen) with 5 μl siPORT NeoFX transfection agent to a final siRNA concentration of 0.375 μM according to the manufacturer's specifications. siRNA complexes were plated and overlaid with 2×10⁵ Huh7.13 cells in antibiotic-free media. The transfection media was replaced with complete media after 24 hours.

Viral Infections and Titering Assays.

Hela^{shS25} or HeLa^{shV} cells were infected with Poliovirus (Mahoney strain) at an MOI of 0.1 in CPBS (137 mM NaCl, 2.7 mM KCl, 10 mM Na₂HPO₄, 2 mM potassium phosphate at pH 7.4, 0.1 mg/mL CaCl₂, 0.1 mg/mL MgCl₂). Infections were carried out for 30 minutes at 37° C. and 5% CO₂, by rolling the plates every 10 minutes. The virus was removed and complete media was added. At 6 hours post infection, the media was removed and the cells were scraped in phosphate buffered saline (PBS: 137 mM NaCl, 2.7 mM KCl, 10 mM sodium phosphate dibasic, 2 mM potassium phosphate at pH 7.4). Virus was isolated by 3 freeze thaw cycles, then the debris was pelleted and the supernatant containing the virus was tittered. Briefly, 10-fold serial dilutions of the virus were used to innoculate HeLa cells. The inoculum was removed after 30 minutes and 2 ml of agarose (1%0 agarose, 1×199 media. 10% FBS. 12 mM HEPES. 0.2% NaHCO₃, 1% penicillin/streptomycin, 1% L-glutamine) was overlayed onto the HeLa cells. After 36 hours, cells were fixed with 10% TCA and stained with 1% crystal violet and plaques were counted.

Hela^{shS25} or HeLa^{shV} cells were infected with Herpes simplex virus 1 strain F (HSV-1) at an MOI of 0.1 and incubated. One round of infection was carried out at 37° C. and 5% CO₂. After 24 hours, the media was removed and the virus was harvested in sterile milk. Viral titer was determined as described above for poliovirus.

Hela^{shS25} or HeLa^{shV} cells were infected with Adenovirus serotype 5 (Ad5) at an MOI of 0.1. Infections were carried out at 37° C. and 5% CO₂ for 30 hours (one round of infection). The media was removed and virus was harvested by scraping in PBS. Virus was isolated by 3 freeze thaw cycles, then the debris was pelleted and the supernatant containing the virus was tittered as described above except that 911 cells (Ad5 E1-transformed human embryonic retina cells (Fallaux et al., Human Gene Therapy 7:215-22 (1996)) were used and plaques were counted on day 8.

The HCV infection was carried out by inoculating Huh7.13 cells with 1 ml of culture medium from the HCV producer cell line Huh7 JFH1 HCV (genotype 2a). Media was replaced after two hours and the cells were incubated for 3 days at 37° C. prior to analysis. Cells were lysed 72 hours post infection and a western analysis was performed using the NS5A antibody.

Northern Analysis.

Total RNA was harvested with TRIzol (Invitrogen) according to the manufacturer's protocol from shRNA lentiviral transduced cells. 5 μg of RNA or 5 μl of RNA extracted from polysome fractions, were separated on a denaturing agarose gel (0.8% agarose, 16% formaldehyde) in MOPS buffer (20 mM MOPS, 5 mM NaOAc, 1 mM EDTA at pH7.0) and transferred to a zeta-probe membrane (Bio-Rad; Hercules, Calif.). ³²P-dCTP (PerkinElmer) radiolabeled probes were generated with the Prime-a-gene kit (Promega) using PCR products amplified from a HeLa cDNA pool with primer sets P5-P7 for RPS25, β-Actin and Cyclin Ti. The p53, probe template was made using the IRES sequence, by digesting pΔEMCV-p53 with EcoRI-NcoI.

Western Analysis.

Cells were lysed in E1 lysis buffer with 0.1% SDS. 20 μg of each lysates was separated on a 12 or 15% SDS-PAGE gel. Following electrophoresis, proteins were transferred to a polyvinylidene difluoride membrane, Immobilon-P (Millipore Co.; Milford, Mass.), using the genie wet transfer system (Idea Scientific Company; Minneapolis, Minn.). Membranes were incubated in blocking buffer (3% dry milk in PBST (PBS with 1% Tween-20)) for 1 hour at room temperature and incubated with primary antibody diluted in blocking buffer overnight at 4° C. Membranes were washed three times in PBST and secondary antibody was applied for one hour in blocking buffer using a 1:5000 dilution. A rabbit polyclonal RPS25 antibody was generated using a His-tagged recombinant RPS25 protein by PrimmBiotech (Cambridge, Mass.) and used at a 1:200 dilution. The commercial antibodies used were: β-Actin (Santa Cruz, #sc-47778) (Santa Cruz Biotechnology; Santa Cruz, Calif.) at 1:2000 dilution, and NS5A antibody. Signals were detected using flororchrome-conjugated secondary antibody for quantitative western blotting using the Odyssey scanner and software (LI-COR; Lincoln, Nebr.).

Luciferase Assay.

Cells from a 24 well plate were washed with PBS and lysed directly in the plate with 100 μl of 1× passive lysis buffer (Promega). In the adenovirus shunting experiments. 5×10⁴ HeLa cells were transfected with the B202 shunting reporter and β-galactosidase reporter. The cells were allowed to recover from transfection for 24 hours and then were infected with Ad5 at an MOI of 25. After one round of infection the cells were processed for the luminescence assays. 4 μl of lysate were assayed using a FB 12 luminometer (Berthold Technologies USA; Oak Ridge, Tenn.) according to the manufacturer's instructions for the dual luciferase kit (Promega). All assays were performed in triplicate.

β-Galactosidase Assay.

Cells from a 24 well plate were washed with PBS and lysed directly in the plate with 100 μl of lysis buffer (100 mM potassium phosphate pH 7.8, 0.2% Triton X-100 (Applied Biosystems: Carlsbad, Calif.)). The β-galactosidase activity of each lysate was measured using the Galacto-Light Plus kit according to the manufacturer's instructions (Applied Biosystems). Briefly. 20 μl of lysate were incubated with 200 μl of 1× chemiluminescent substrate in reaction buffer (100 mM NaPO₄ pH 8.0, 1 mM MgCl₂). After incubation at room temperature for one hour, 300 μl of Accelerator-II was added and the luminescence was measured using a FB 12 luminometer (Berthold). All assays were performed in triplicate.

Polysome Analysis.

Approximately 2×10⁷ cells were grown to 70% confluency. Cycloheximide (0.1 mg/ml final concentration) was added to the medium for 3 minutes at 37° C. to arrest the ribosomes. The cells were washed with PBS containing 0.1 mg/ml cycloheximide and then lysed for 10 minutes on ice with 400 μl polysome extraction buffer (15 mM Tris-Cl, ph 7.4, 15 mM MgCl₂, 0.3 M NaCl. 0.1 mg/ml cycloheximide, 1 mg/ml heparin, 1% Triton X-100). The lysates were cleared by centrifugation at 13,200×g for 10 minutes. 500 μl of the supernatant was layered on 20-50% sucrose gradients, which contained polysome extraction buffer with no Triton X-100. The gradients were sedimented at 151.263×g for 190 minutes in a SW41 rotor at 4° C. An ISCO UA-5 fraction collection system (Teledyne; Thousand Oaks, Calif.) was used to collect 14 fractions that were immediately mixed with 2 ml of 8M guanidine HCl.

Total RNA were precipitated from polysome fractions by ethanol precipitation and dissolved in 25 μl of H₂O based on common protocols (Johannes et al., Proc. Natl. Acad. Sci. USA 96:13118-23 (1999)). Briefly, guanidine containing samples were vortexed for 20 seconds. 3 ml of 100% ethanol was added and the fraction was vortexed again. The faction was incubated overnight at −20° C. to allow for complete precipitation of the RNA. The fractions were centrifuged at 14462×g in a SS-34 rotor for 30 minutes at 4° C. The RNA pellet was washed with 75% ethanol. The pellet was resuspended in 400 μl 1×TE pH 8.0 and transferred to a microcentrifuge tube containing 100 μM NaOAc pH 5.3 and 1 ml ethanol and incubated overnight at −20° C. to precipitate RNA. RNA precipitation and wash steps were performed as before and allowed to dry before the final re-suspension in 25 μl of H₂O. One fifth (5 μl) of the total RNA from each fraction was separated on a denaturing RNA gel and probed for a specific mRNA as indicated in the northern analysis section.

### Results

### Example 1

### RPS25 is not Essential for Mammalian Cells in Culture

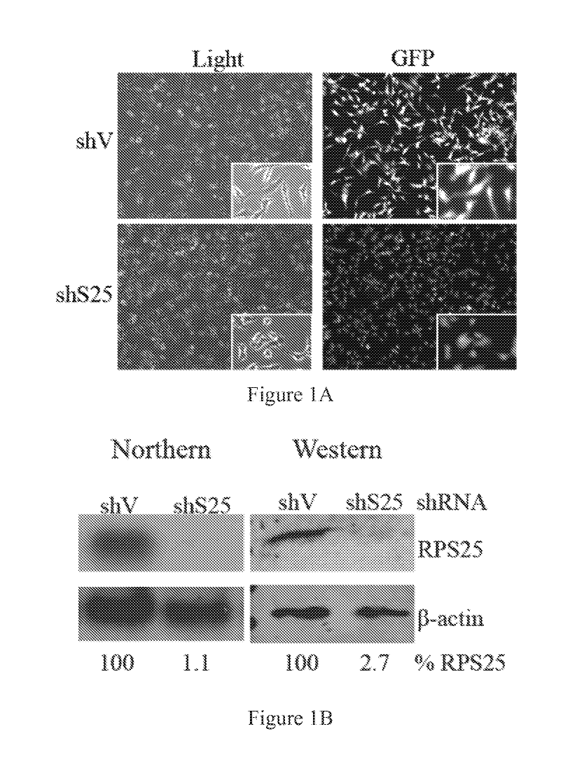

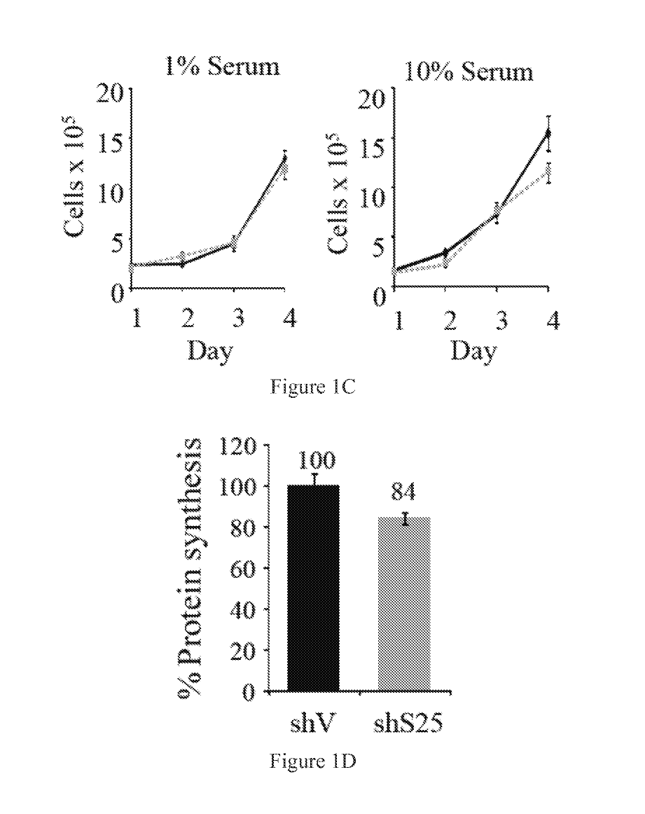

To determine if RPS25 is essential in mammalian cells, stable HeLa cells lines were generated that have RPS25 stably knocked down by transducing them with a lentivirus that either expressed an shRNA against RPS25 (HeLa^{shS25}) or not (HeLa^{shV}). Stably transduced cells were isolated by cell sorting based on expression of the green fluorescent protein (GFP) from the lentiviral vector (FIG. 1A). RPS25 mRNA and protein expression levels were undetectable in HeLa^{shS25} cells (FIG. 1B) demonstrating that RPS25 is efficiently knocked down. The HeLa^{shS25} cell line was further characterized to determine if there were any gross defects due to prolonged knockdown of RPS25. The HeLa^{shS25} cells were morphologically similar to control, HeLa^{shV}, cells (FIG. 1A) and there was no significant defect in growth rate except at the highest serum concentration (FIG. 1C). There was a small decrease in protein synthesis rates for the HeLa^{shS25} cells at 10% serum as well. These findings are consistent with our previous findings in yeast (Landry et al., Genes Dev. 23:2753-64 (2009)). Taken together, these results show that RPS25 is not essential in mammalian cells but may confer a slight growth advantage to rapidly growing cells.

### Example 2

### IRES-Mediated Translation is Defective in HeLa^{shS25} Cells

To determine if IRES-mediated translation is affected by stable knockdown of RPS25, the CrPV IGR IRES activity was measured in the HeLa^{shS25} cells using a bicistronic reporter assay (FIG. 2A). The CrPV IGR IRES activity in the RPS25 depleted cells is 10-13% of wild-type activity (FIG. 2B, compare black and dark gray bars), which is consistent and slightly lower than the decrease in IRES activity observed with transient knockdown of RPS25 (Landry et al., Genes Dev. 23:2753-64 (2009)). To verify that the HeLa^{shS25} cell line did not accumulate additional confounding mutations that could potentially affect IRES activity, whether IRES activity could be rescued by expression of an shRNA resistant RPS25 was tested (FIG. 2A). Since the RPS25 shRNA targeted the coding region of RPS25, an shRNA-resistant RPS25 expression plasmid, hS25, was generated, with synonymous mutations in the siRNA recognition motif (FIG. 2A). Transient expression of hS25 in the HeLa^{shS25} cells resulted in an increase in RPS25 protein expression after 24 hours that was maintained for at least 72 hours demonstrating that hS25 from the rescue plasmid was resistant to the shRNA (FIG. 2C). Expression of hS25 resulted in a partial restoration of CrPV IGR IRES activity 24 hours post transfection and a complete restoration by 48 hours (FIG. 2B, white bars). The fact that exogenous expression of an shRNA resistant RPS25 rescued IRES activity suggests that the only factor missing for IRES-mediated translation in HeLa^{shS25} s cells is RPS25.

### Example 3

### Viral IRESs that are Structurally and Functionally Different Rely on RPS25

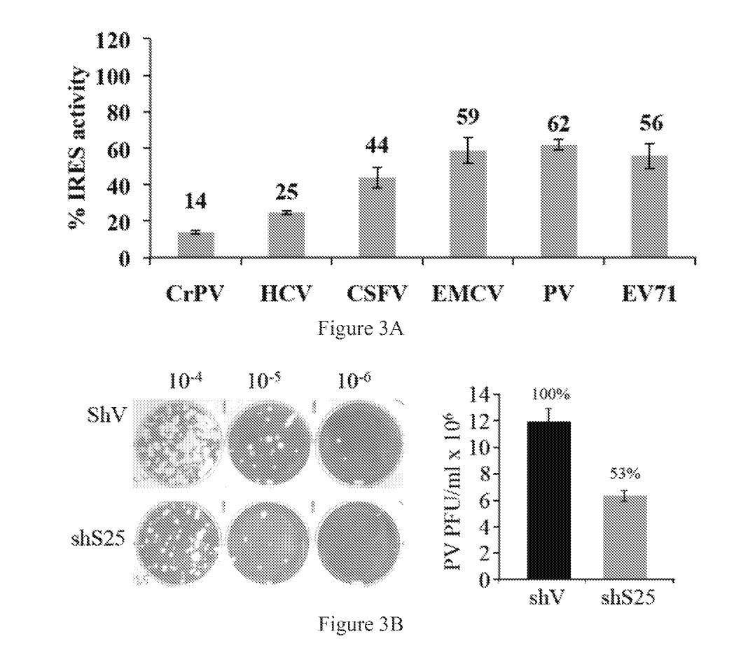

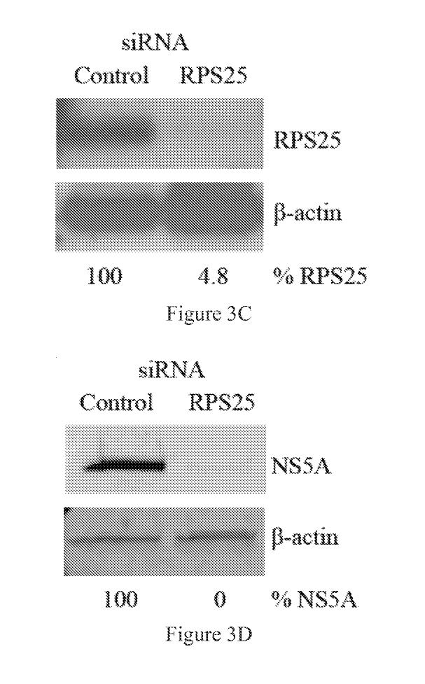

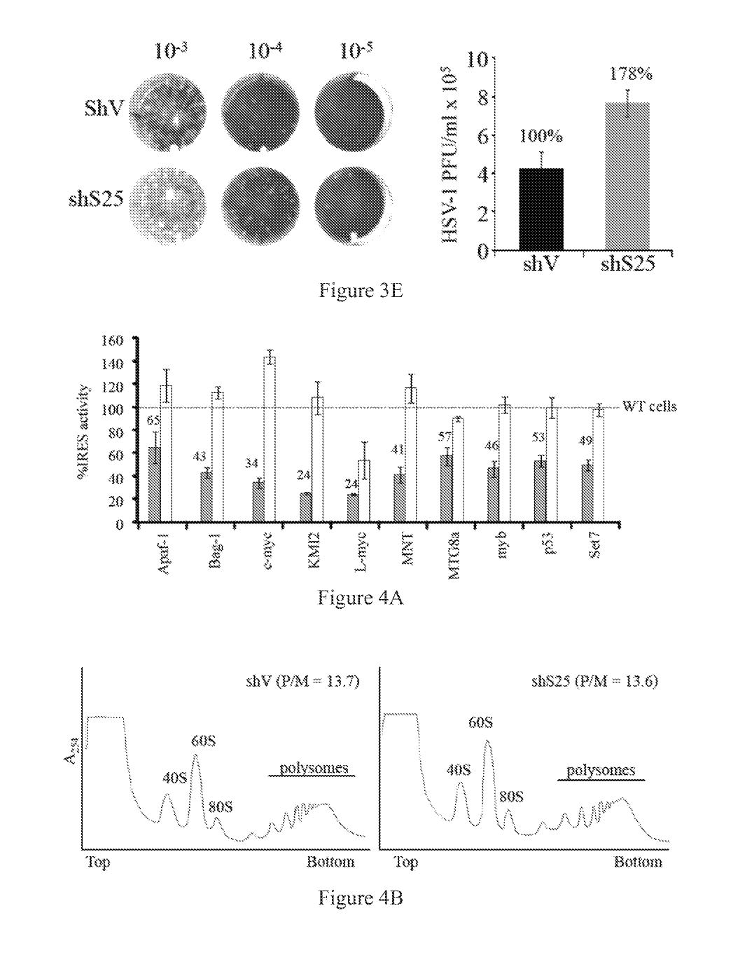

To determine whether RPS25 is required by other viral IRESs, the IRES activity for a range of viral IRESs was tested in the HeLa^{shS25} cell line (FIG. 3A). HCV and classic swine fever virus (CSFV) are both members of the Flaviviridae virus family and have similar IRES elements (Kolupaeva et al., J. Virol. 74:6242-50 (2000)). The activity of the HCV IRES is decreased to 25% in the HeLa^{shS25} cells in agreement with results obtained after a transient knockdown of RPS25 in HeLa cells (Landry et al., Genes Dev. 23:2753-64 (2009)). The CSFV IRES activity was reduced to 44% in the HeLa^{shS25} cells demonstrating that other flaviviral IRESs also use RPS25-driven translation (FIG. 3A).

Both the Dicistroviridae and Flaviviridae IRESs are known to recruit the 40S ribosomal subunit in the absence of any initiation factors and they both recruit the ribosome directly to the start codon. To examine whether other types of IRESs that require some subset of initiation factors to recruit the 40S subunits also require RPS25, the activity of various picornaviral IRESs were determined when RPS25 was knocked down. The encephalomyocarditis virus (EMCV) IRES recruits the ribosome directly to the AUG start codon (Hellen and Sarnow, Genes Dev. 15:1593-1612 (2001)). In contrast, the IRES elements in poliovirus (PV) and enterovirus 71 (EV71) recruit the ribosome upstream of the start codon and the ribosome scans down to the start codon (Thompson and Sarnow, Virology 315:259-66 (2003)). Interestingly, both types of picornaviral IRESs are equally compromised in the HeLa^{shS25} cells (FIG. 3A) indicating that RPS25 has a role in picornaviral IRESs that is independent of ribosome scanning.

### Example 4

### RPS25 is Required for the Amplification of IRES-Containing Viruses

Many viruses, such as HCV and PV, rely solely on IRES-mediated translation to generate viral proteins, which suggests that depletion of RPS25 would inhibit viral replication. Therefore, the replication of PV and HCV were assayed in RPS25 depleted cells. Both HeLa^{shS25} and control cells (HeLa^{shV}) were infected with PV and the amount of virus produced from a single round of replication was determined by plaque assay (FIG. 3B). There was a 47% reduction in PV titer in RPS25 knockdown cells demonstrating that the decrease in PV IRES activity from knockdown of RPS25 results in a decrease in PV amplification (FIG. 3B).

HCV replicates efficiently in Huh7 cell lines (Cai et al., J. Virol. 79:13963-73 (2005)). Therefore, to determine whether RPS25 is required for HCV amplification, an Huh7.13 cell line was transiently transfected with an siRNA that targets RPS25. The RPS25 knockdown was over 95% effective (FIG. 3C). These cells were infected with HCV at a low MOI (multiplicity of infection) such that amplification of the virus is required in order to detect viral proteins by western (Cun et al., J. Virol. 84:11532-41 (2010)). In the presence of a non-targeting control siRNA, there was robust expression of the nonstructural protein, NS5A, 72 hours after infection (FIG. 3D). However, in contrast the HCV NS5A was not detected in the RPS25 knockdown Huh7.13 cells, demonstrating that RPS25 is required for amplification of HCV in cell culture (FIG. 3D).

Without intending to be limited by theory, it is believed that the reduction in viral amplification is due to a decrease in viral protein production when RPS25 levels are reduced. Accordingly, a virus that does not use an RPS25-dependent IRES should not have a defect in replication in the HeLa^{shS25} cell line. To test this, the effect of knocking down RPS25 on a DNA virus, herpes simplex virus 1 strain F (HSV-1) was determined. HSV-1 uses a cap-dependent mechanism to translate viral proteins (Smith et al., Biochem. Soc. Trans. 36:701-7 (2008)) and therefore should be unaffected by RPS25 depletion. Viral titers were determined after a single round of infection in HeLa^{shS25} or HeLa^{shV} cells (FIG. 3E). Unlike for HCV and PV, a reproducible 1.5 to 2-fold increase in HSV-1 titers in the HeLa^{shS25} cells was observed. This suggests that there is not a decrease in the fitness of the HeLa^{shS25} cells that causes a non-specific decrease in viral titers. Taken together, this suggests that the reduced viral titers are due to impaired translation of IRESs or ribosomal shunting and not due to a decrease in cell fitness.

### Example 5

### RPS25 Aids in the Translation of Cellular IRESs

To examine whether RPS25 is also required for cellular IRES-mediated translation, several cellular IRESs were assayed for RPS25 dependence in the HeLa^{shS25} and HeLa^{shV} cells using a bicistronic reporter assay. All of the cellular IRESs demonstrated a dependence on RPS25 (FIG. 4A, gray bars). Importantly, IRES activity was rescued by hS25 (FIG. 4A, white bars).

To examine the translational efficiency of an endogenous IRES-containing mRNA, a polysome analysis was performed on lysates from HeLa^{shS25} and HeLa^{shV} cells. The polysome profiles demonstrate that the global translation profiles and the polysome to monosome (P/M) ratios are similar for both HeLa^{shS25} and HeLa^{shV} cells indicating that there are no differences in global translation (FIG. 4B). Furthermore, the size of the 40S peaks are equivalent suggesting that there is no defect in ribosomal subunit production in agreement with the finding in yeast where it was demonstrated that there was no significant defect in rRNA biogenesis in yeast harboring a deletion in RPS25 (Landry et al., Genes Dev. 23:2753-64 (2009)). Last, there is no increase in free ribosomal subunits, which indicates that there is no defect in translation initiation or subunit joining.

To visualize the relative translation efficiencies of specific messages, northern analysis was performed on RNA extracted from the polysome fractions. The β-Actin mRNA was associated with high molecular weight polysomes in both cell lines demonstrating that cap-dependent translation was unaffected by the loss of RPS25 (FIG. 4C). In contrast, a subset of the p53 mRNA accumulated in the 40S peak in the HeLa^{shS25} cells indicating a block in initiation (FIG. 4C). This suggests that the endogenous p53 IRES can bind to 40S subunits, but is blocked at a downstream step. Many IRES-containing cellular RNAs can also be translated by both cap-dependent and cap-independent mechanisms. Consistent with this, there appears to be a population of p53 mRNAs that are translated through a cap-dependent mechanism and remain associated with the polysomes in the absence of RPS25.

### Example 6

### RPS25 is Required for Ribosome Shunting in Adenovirus

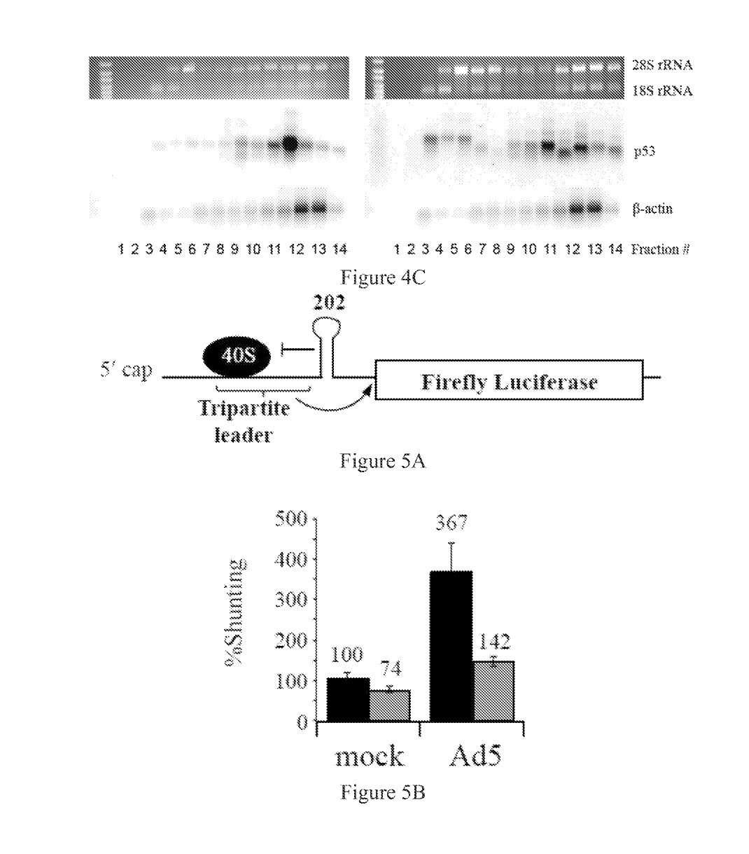

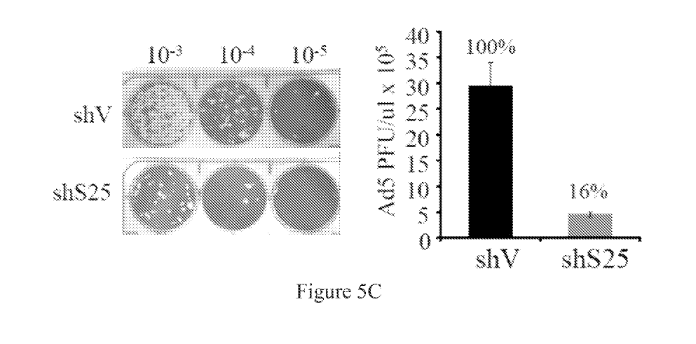

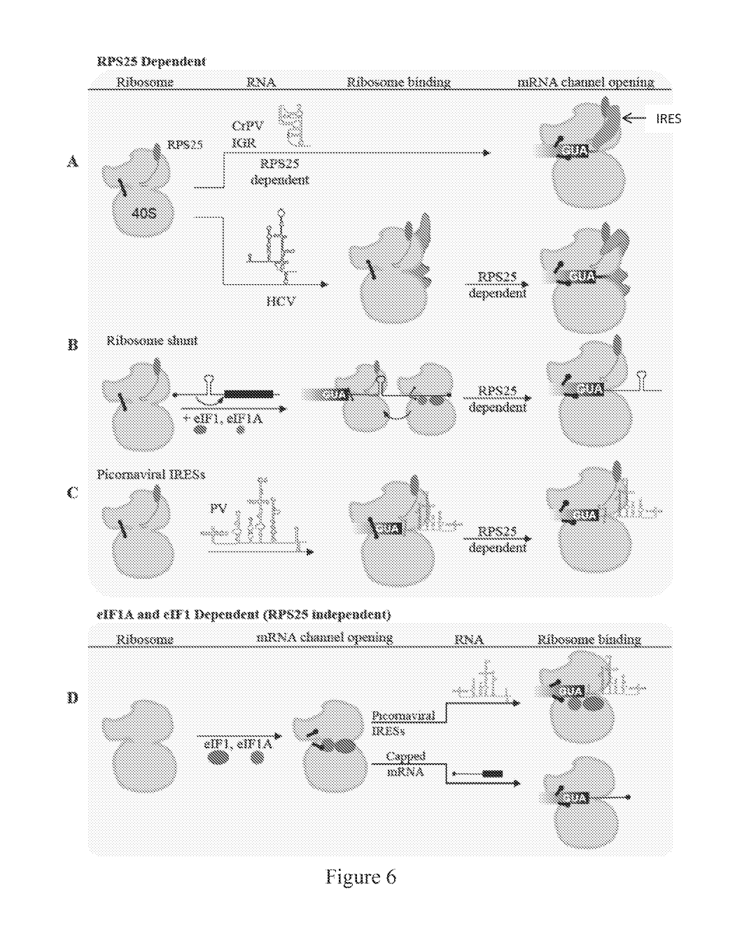

Ribosome shunting is a process in which the ribosome is recruited in a cap-dependent manner and the 40S ribosome bypasses a region of secondary structure in the 5′ UTR without scanning through it, rather the ribosome is shunted to a downstream site to initiate protein synthesis. This strategy has been described for cauliflower mosaic virus and adenovirus and is also used in other viral and cellular mRNAs (Futterer et al., EMBO J. 9:1697-707 (1990); Futterer et al., Cell 73:789-802 (1993); Yueh and Schneider, Genes Dev. 10:1557-67 (1996); Yueh and Schneider, Genes Dev. 14:414-21 (2000); Sherrill and Lloyd, Mol. Cell. Biol. 28:2011-22 (2008)). A shunting luciferase reporter was used to establish whether ribosome shunting was specifically impaired in RPS25 deficient cells. The Ad-hp-luc shunting reporter contains the tripartite leader shunting sequence from the 5′ UTR of adenovirus with a thermodynamically stable stem loop engineered immediately downstream to eliminate scanning through the leader region (FIG. 5A) (Yueh and Schneider, Genes Dev. 10:1557-67 (1996)). In HeLa^{shS25} cells, shunting by the adenovirus tripartite leader exhibited a modest reduction in shunting (26%) (FIG. 5C, mock infected). However, shunting is known to be upregulated during infection (Yueh and Schneider, Genes Dev. 10:1557-67 (1996)), therefore the efficiency of shunting in the absence of RPS25 following infection in HeLa^{shS25} cells was tested. In infected HeLa^{shV} cells shunting increased by 3.7-fold over mock infected cells. In contrast, shunting in infected HeLa^{shS25} increased by only 1.9-fold. Therefore, shunting was reduced by 60% in infected HeLa^{shS25} cells compared to infected control cells (FIG. 5B). Next, the necessity of RPS25 was tested for adenovirus replication by measuring the titer of adenovirus type 5 (Ad5) produced following one round of infection. The Ad5 titer was more than 5-fold lower in the HeLa^{shS25} cells indicating that RPS25 is important for the replication of adenovirus (FIG. 5C).

### Example 7

### RPS25 is Required for Amplification of Dengue Virus and Yellow Fever Virus

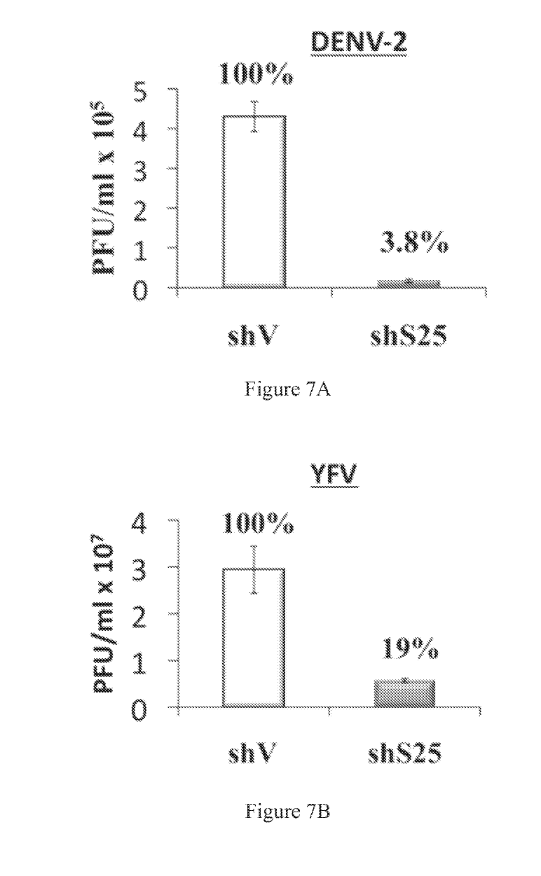

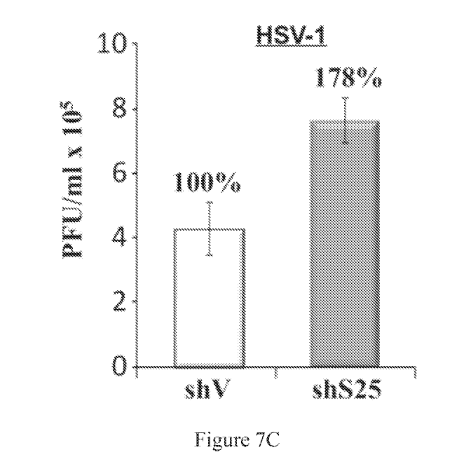

Dengue Virus (DENV) and Yellow Fever Virus (YFV) use a mechanism of initiating translation that is not well understood, however it is clear that these viruses employ a noncanonical translation mechanism (Edgil et al. 2006), which is different from that of the translation of the vast majority of cellular mRNAs. Stable cell lines that express a short hairpin RNA (shRNA) to deplete RPS25 resulted in the knockdown of RPS25. Knockdown of RPS25 resulted in a significant decrease in amplification of YFV and DENV (FIGS. 7A and 7B). Importantly, depletion of RPS25 did not impair viral amplification of Herpes Simplex Virus-1 (HSV-1) (FIG. 7C), a virus that relies on cap-dependent translation. This demonstrates that cells depleted of RPS25 are not intrinsically impaired in their ability to replicate virus, and suggests that RPS25 is a specific target for viruses that use an alternative mechanisms to initiate translation.

1. A method of treating or preventing a viral infection in a subject, the method comprising:
(a) identifying a subject with or at risk of developing a viral infection, wherein the viral infection is mediated by a virus comprising one or more viral RNAs that are translated by a ribosomal shunting mechanism or a non-IRES mediated mechanism;
(b) administering to the subject a therapeutically effective amount of an agent that reduces ribosomal protein S25 (Rps25) expression or function in the subject in comparison to a control.

2-10. (canceled)

11. The method of claim 1, wherein the agent is a compound of the following formula: 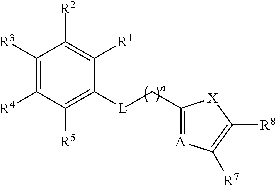
or a pharmaceutically acceptable salt of prodrug thereof, wherein:
A is CR⁹ or N;
L is —O—CR¹⁰R¹¹C(O)—NR⁶—, —NR¹²—NR⁶—, —C(O)—NR⁶—, —SO₂—NR⁶—, —CH₂—NR⁶—, —CH₂—CH₂—NR⁶—, or a substituted or unsubstituted heteroaryl;
n is 0, 1, or 2;
X is —CR¹³═CR¹⁴—, —N═CR¹⁵—, —CR¹⁵═N—, NR¹⁶, O, or S;
R¹, R², R³, R⁴, R⁵, R⁷, R⁸, R⁹, R¹⁰, R¹¹, R¹³, R¹⁴, and R¹⁵ are each independently selected from hydrogen, halogen, hydroxyl, trifluoromethyl, substituted or unsubstituted thio, substituted or unsubstituted alkoxyl, substituted or unsubstituted aryloxyl, substituted or unsubstituted amino, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl; and
R⁶, R¹², and R¹⁶ are each independently selected from hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl, or substituted or unsubstituted carbonyl.

12. The method of claim 11, wherein R¹ and R² are combined to form a substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl.

13. The method of claim 11, wherein R² and R³ are combined to form a substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl.

14. The method of claim 11, wherein R³ and R⁴ are combined to form a substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl.

15. The method of claim 11, wherein R⁵ and R⁶ are combined to form a substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl.

16. The method of claim 11, wherein A is CH or N.

17. The method of claim 11, wherein L is a substituted or unsubstituted pyrazole.

18. The method of claim 11, wherein R³ is ethoxy, dimethylamino, or chloro.

19. The method of claim 11, wherein X is S or —CH═CH—.

20.-32. (canceled)

33. A method of inhibiting ribosomal shunting-mediated translation or non-IRES mediated translation, the method comprising:
(a) providing a cell, wherein the cell comprises an RNA molecule that is translated by a ribosomal shunting mechanism or a non-IRES mediated mechanism; and
(b) contacting the cell with an agent that reduces ribosomal protein S25 (Rps25) expression or function, reduction of Rps25 expression or function as compared to a control indicates that the agent inhibits ribosomal shunting-mediated translation or non-IRES mediated translation.

34. The method of claim 33, further comprising determining that ribosomal shunting-mediated translation non-IRES mediated translation is inhibited by detecting a reduced level of protein expressed by an RNA translated by the ribosomal shunting mechanism or non-IRES mediated mechanism in comparison to a control.

35.-42. (canceled)

43. A method of screening for an agent that inhibits or promotes ribosomal shunting-mediated translation or non-IRES mediated translation, the method comprising:
(a) providing a system comprising a ribosomal protein S25 (Rps25) or a nucleic acid that encodes Rps25 and an RNA molecule translated by a ribosomal shunting mechanism or a non-IRES mediated mechanism;
(b) contacting the system with the agent to be screened; and
(c) determining Rps25 expression or function, wherein a decrease in the level of Rps25 expression or function indicates the agent inhibits ribosomal shunting-mediated translation or non-IRES mediated translation, and wherein an increase in the level of Rps25 expression or function indicates the agent promotes ribosomal shunting-mediated translation or non-IRES mediated translation.

44. The method of claim 43, wherein the system comprises a cell.

45. The method of claim 43, wherein the system comprises an in vitro assay.

46. The method of claim 43, wherein the agent to be tested is selected from the group consisting of a small molecule, a polypeptide, a nucleic acid molecule, a peptidomimetic, or a combination thereof.

47. (canceled)

48. A method of identifying cellular RNA molecules translated by a ribosomal shunting mechanism or a non-IRES mediated mechanism, the method comprising:
(a) inhibiting Rps25 expression or function in a cell;
(b) determining a protein expression pattern in the cell; and
(c) comparing the protein expression pattern in the cell to a control, wherein a decrease in protein expression of a cellular RNA molecule as compared to a control indicates the cellular RNA molecule is translated by a ribosomal shunting mechanism or non-IRES mediated mechanism.

49. A method of promoting ribosomal shunting-mediated translation or non-IRES mediated translation, the method comprising:
(a) providing a cell, wherein the cell comprises an RNA molecule translated by a ribosomal shunting mechanism or a non-IRES mediated mechanism; and
(b) contacting the cell with an agent that increases ribosomal protein S25 (Rps25) expression or function, wherein an increase in Rps25 expression or function as compared to a control indicates that the agent promotes ribosomal shunting-mediated translation or non-IRES mediated translation.

50. The method of claim 49, further comprising determining that ribosomal shunting-mediated translation or non-IRES mediated translation is promoted by detecting an increased level of protein encoded by the RNA molecule translated by a ribosomal shunting mechanism or a non-IRES mediated mechanism in comparison to a control.

51. A method of promoting ribosomal shunting-mediated translation or non-IRES mediated translation, the method comprising providing a cell with a nucleic acid encoding a Rps25 protein or functional fragment thereof.
